(19) United States
(12) Patent Application Publication
(10) Pub. No.: US 2020/0196434 A1
(43) Pub. Date: Jun. 18, 2020

(54) ENDOSCOPE DEVICE, AND FLEXIBLE PRINTED CIRCUIT BOARD ASSEMBLY AND FLEXIBLE CIRCUIT BOARD THEREOF

(71) Applicant: ALTEK BIOTECHNOLOGY CORPORATION, Hsinchu City (TW)

(72) Inventors: FANG-MENTG KUO, HSINCHU CITY (TW); TE-YU CHUNG, HSINCHU CITY (TW); CHAO-YU CHOU, HSINCHU CITY (TW)

(21) Appl. No.: 16/711,518

(22) Filed: Dec. 12, 2019

(30) Foreign Application Priority Data
Dec. 12, 2018 (TW) 107144790
Nov. 7, 2019 (TW) 108140527

(51) Int. Cl. H05K 1/02; G02B 23/24; H04N 5/225; A61B 1/018; A61B 1/06; A61B 1/00; A61B 1/04; A61B 1/005

(52) U.S. Cl. CPC H05K 1/0203; H04N 2005/2255; G02B 23/2484; G02B 23/2461; H04N 5/2257; A61B 1/018; A61B 1/06; A61B 1/00124; A61B 1/04; A61B 1/005; H05K 2201/056; H05K 2201/10121; H05K 2201/10106; H05K 2201/10189; H05K 2201/2018; H05K 2201/10356; H05K 1/028

(57) ABSTRACT

An endoscope device, a flexible printed circuit board assembly and a flexible printed circuit board thereof are provided. The flexible printed circuit board includes a sensing module carrier portion, a first body portion, a second body portion, a third body portion, a fourth body portion and a light-emitting element carrier portion. The first body portion and the second body portion are respectively located on opposite sides of the sensing module carrying portion. The fourth body portion is connected with the second body portion, and the third body portion and the fourth body portion are respectively located on opposite sides of the sensing module carrying portion. The light-emitting element carrier portion includes a first carrier side portion connected to the third body portion and a second carrier side portion connected to the third body portion. The first and the second carrier side portions are separated from each other.

This application claims the benefit of priority to Taiwan Patent Application No. 107144790, filed on Dec. 12, 2018 and Taiwan Patent Application No. 108140527, filed on Nov. 7, 2019. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an endoscope device, a flexible printed circuit board assembly and a flexible printed circuit board thereof, and more particularly to an endoscope device, a flexible printed circuit board assembly and a flexible printed circuit board capable of improving heat dissipation efficiency.

### BACKGROUND OF THE DISCLOSURE

With the development of endoscope technology, functional requirements of endoscopes are increasing, which has resulted in an increase in the number of requisite electronic components in the endoscope. However, when a large number of electronic components are disposed in the endoscope, it is necessary to consider not only the arrangement positions of the electronic components, the cable management and the soldering work, but also the heat dissipation efficiency of the electronic components.

Therefore, how the above defects can be overcome through the improvement of structural design has become one of the important topics in the related art.

### SUMMARY OF THE DISCLOSURE

In response to the above-referenced technical inadequacies, the present disclosure provides an endoscope device, a flexible printed circuit board assembly and a flexible printed circuit board thereof.

In one aspect, the present disclosure provides an endoscope device including: a hollow tube and a flexible printed circuit board assembly. The flexible printed circuit board assembly is disposed in the hollow tube, and includes a carrier base, a flexible printed circuit board, an image sensing module, an light-emitting element, a cable module, and a supporting frame. The flexible printed circuit board is disposed on the carrier base, and the image sensing module is coupled to the flexible printed circuit board and disposed on a sensing module carrier portion of the flexible printed circuit board. The light-emitting element is coupled to the flexible printed circuit board and disposed on an light-emitting element carrier portion of the flexible printed circuit board, the cable module is coupled to the flexible printed circuit board and disposed on the flexible printed circuit board, and the supporting frame is configured to support the carrier base and the Light-emitting element carrier portion. The flexible printed circuit board is disposed in a curved shape on the carrier base, and an accommodating space is defined between the flexible printed circuit board and the carrier base. A connector of the cable module is coupled to the flexible printed circuit board and disposed in the accommodating space. The flexible printed circuit board includes a sensing module carrier portion, a first body portion, a second body portion, a third body portion, a fourth body portion and a light-emitting element carrier portion, the first body portion is connected to one side of the sensing module carrier portion, the second body portion is connected to the other side of the sensing module carrier portion and corresponds to the first body portion, the third body portion is connected to the first body portion, the fourth body portion is connected to the second body portion and the light-emitting element carrier portion is connected to the third body portion. The accommodating space is defined between the fourth body portion and the carrier base, and the connector of the cable module is disposed on the fourth body portion and located in the accommodating space.

In one aspect, the present disclosure provides a flexible printed circuit board assembly, including: a carrier base, a flexible printed circuit board, an image sensing module, a light-emitting element, and a cable module. The flexible printed circuit board is disposed on the carrier base, and the flexible printed circuit board includes a sensing module carrier portion, a first body portion, a second body portion, a third body portion, and an light-emitting element carrier portion. The first body portion is connected to one side of the sensing module carrier portion, the second body portion is connected to the other side of the sensing module carrier portion and corresponds to the first body portion, the third body portion is connected to the first body portion, and the light-emitting element carrier portion is connected to the third body portion. The image sensing module is coupled to the flexible printed circuit board and disposed on the sensing module carrier portion. The Light-emitting element is coupled to the flexible printed circuit board, and disposed on the Light-emitting element carrier portion. The cable module is coupled to the flexible printed circuit board, and includes a connector coupled to the flexible printed circuit board and a plurality of wires disposed on the connector. The flexible printed circuit board is disposed in a curved shape on the carrier base, and an accommodating space is defined between the flexible printed circuit board and the carrier base. The connector of the cable module is disposed in the accommodating space. The flexible printed circuit board further includes a fourth body portion coupled to the second body portion, wherein the accommodating space is defined between the fourth body portion and the carrier base, and the connector of the cable module is disposed on the fourth body portion and located in the accommodating space

In one aspect, the present disclosure provides another flexible printed circuit board, including: a sensing module carrier portion, a first body portion, a second body portion, a third body portion, a fourth body portion, and an light-emitting element carrier portion. The first body portion is coupled to one side of the sensing module carrier portion. The second body portion is coupled to the other side of the sensing module carrier portion and corresponds to the first body portion, and the first body portion and the second body portion are respectively located on opposite sides of the sensing module carrying portion. The third body portion is coupled to the first body portion. The fourth body portion coupled to the second body portion, and the third body portion and the fourth body portion are respectively located on opposite sides of the sensing module carrying portion. The light-emitting element carrier portion is coupled to the third body portion. The light-emitting element carrier portion includes a first carrier side portion connected to the third body portion and a second carrier side portion connected to the third body portion, and the first carrier side portion and the second carrier side portion are separated from each other. The extending directions of the first body portion and the second body portion may be substantially perpendicular to the extending directions of the third body portion and the fourth body portion.

Therefore, one of the beneficial effects of the present disclosure is that the endoscope device, the flexible printed circuit board assembly and the flexible printed circuit board thereof provided by the present disclosure has the technical feature of “the flexible printed circuit board being disposed in a curved shape on the carrier base, an accommodating space being defined between the curved flexible printed circuit board and the carrier base, and the connector of the cable module being coupled to the flexible printed circuit board and disposed in the accommodating space,” so as to improve the heat dissipation efficiency and enable electronic elements to be configured at appropriate positions.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a perspective exploded view of a flexible printed circuit board according to an embodiment of the present disclosure.

FIG. 2 is another perspective exploded view of the flexible printed circuit board according to the embodiment of the present disclosure.

FIG. 3 is a schematic top view of the flexible printed circuit board according to the embodiment of the present disclosure.

FIG. 4 is a perspective assembled view of an endoscope device according to the embodiment of the present disclosure.

FIG. 5 is another perspective assembled view of the endoscope device according to the embodiment of the present disclosure.

FIG. 6 is a perspective exploded view of the endoscope device according to the embodiment of the present disclosure.

FIG. 7 is another perspective exploded view of the endoscope device according to the embodiment of the present disclosure.

FIG. 8 is a perspective exploded view of a flexible printed circuit board assembly according to the embodiment of the present disclosure.

FIG. 9 is another perspective exploded view of the flexible printed circuit board assembly according to the embodiment of the present disclosure.

FIG. 10 is still another perspective exploded view of the flexible printed circuit board assembly according to the embodiment of the present disclosure.

FIG. 11 is a perspective exploded view of another configuration of the flexible printed circuit board assembly according to the embodiment of the present disclosure.

FIG. 12 is a schematic diagram of an assembly process of the flexible printed circuit board and a carrier base according to the embodiment of the present disclosure.

FIG. 13 is another schematic diagram of an assembling process of the flexible printed circuit board and the carrier base according to the embodiment of the present disclosure.

FIG. 14 is still another schematic diagram of an assembling process of the flexible printed circuit board and the carrier base according to the embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

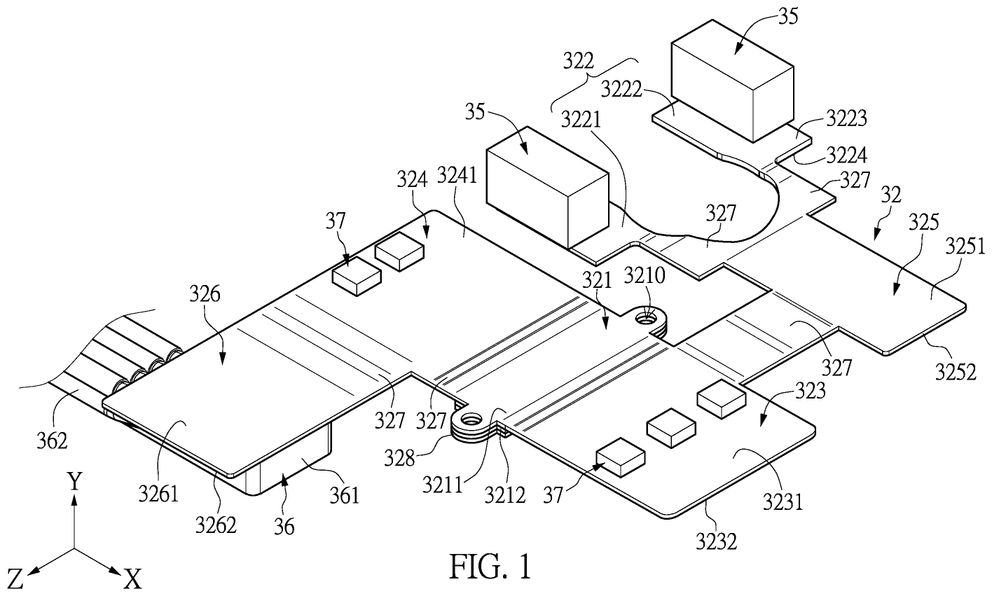

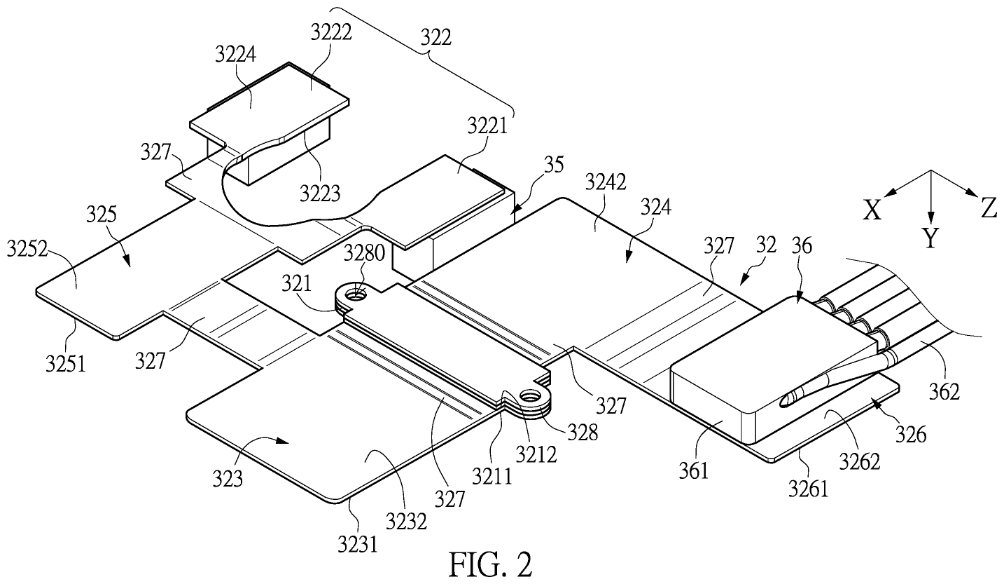

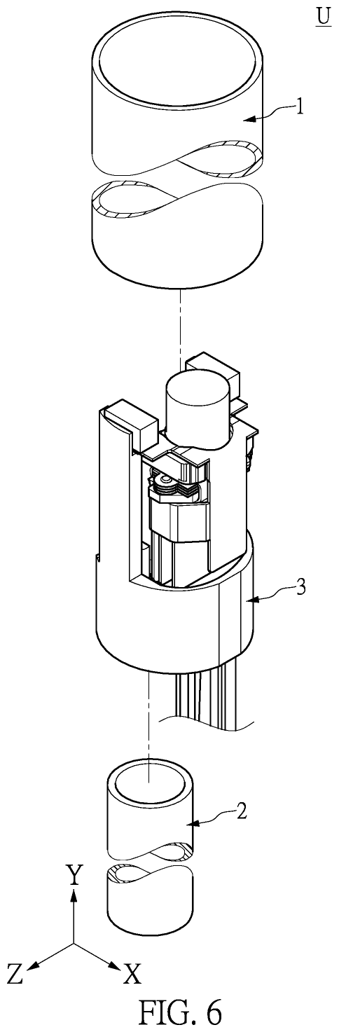

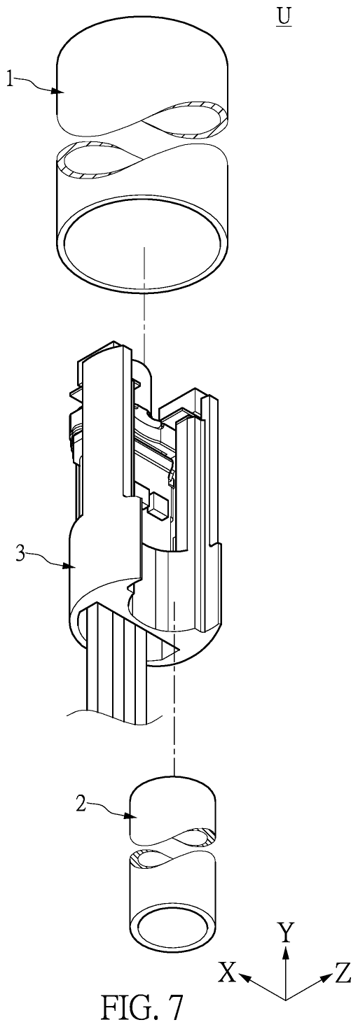

Referring to FIG. 1, FIG. 2, FIG. 6 and FIG. 7, FIG. 1 and FIG. 2 are perspective exploded views of a flexible printed circuit board according to an embodiment of the present disclosure, and FIG. 6 and FIG. 7 are perspective exploded views of an endoscope device according to the embodiment of the present disclosure. The embodiment of the present disclosure provides an endoscope device U, a flexible printed circuit board assembly 3 and a flexible printed circuit board 32 thereof. The flexible printed circuit board 32 can be applied to the flexible printed circuit board assembly 3, and the flexible printed circuit board assembly 3 can be applied to the endoscope device U. However, the present disclosure is not limited thereto. In other words, the flexible printed circuit board 32 and the flexible printed circuit board assembly 3 can also be applied to other electronic devices. The present disclosure is not limited by the application of the flexible printed circuit board assembly 3 and the flexible printed circuit board 32. It should be noted that the main structure of the flexible printed circuit board 32 will first be described in the following, and the more detailed components of the endoscope device U and the flexible printed circuit board assembly 3 will be described later.

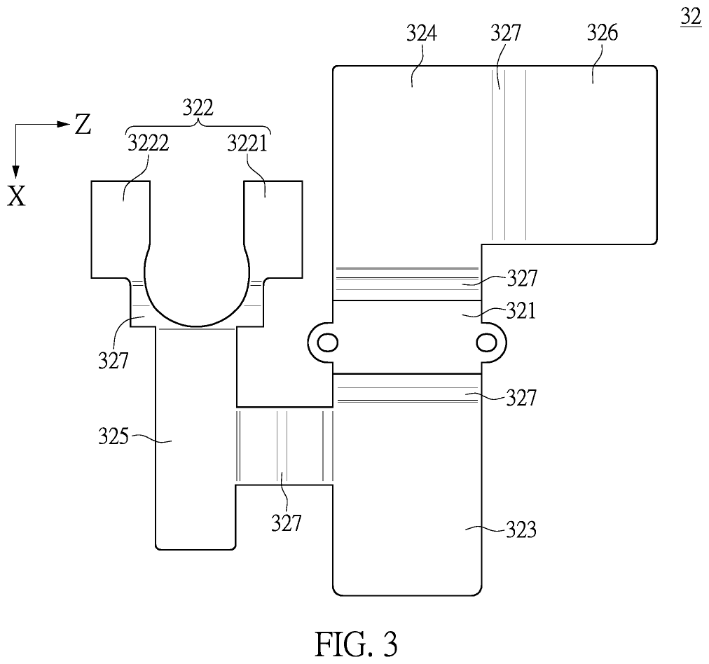

Referring to FIG. 1, FIG. 2, and FIG. 3, FIG. 3 is a schematic top view of the flexible printed circuit board according to an embodiment of the present disclosure. In detail, the flexible printed circuit board 32 can include a sensing module carrier portion 321, an light-emitting element carrier portion 322, a first body portion 323, a second body portion 324, and a third body portion 325. The first body portion 323 is connected to one side of the sensing module carrier portion 321, and the second body portion 324 is connected to the other side of the sensing module carrier portion 321 and correspond to the first body portion 323, in other words, the first body portion 323 and the second body portion 324 are respectively located on opposite sides of the sensing module carrier portion 321. Further, the third body portion 325 may be coupled to the first body portion 323, and the light-emitting element carrier portion 322 may be coupled to the third body portion 325.

In the above embodiment of the present disclosure, the light-emitting element carrier portion 322 may include a first carrier side portion 3221 connected to the third body portion 325 and a second carrier side portion 3222 connected to the third body portion 325, and the first carrier side portion 3221 and the second carrier side portion 3222 are separated from each other. Moreover, in the embodiment of the present disclosure, the flexible printed circuit board 32 may further include a fourth body portion 326 to which the second body portion 324 may be connected. In addition, as shown in FIG. 1 to FIG. 3, the extending directions of the first body portion 323 and the second body portion 324 may be substantially perpendicular to the extending directions of the third body portion 325 and the fourth body portion 326. That is, with reference to an XYZ axis, the extending direction of the first body portion 323 and the second body portion 324 can be along the X direction, while the extending direction of the third body portion 325 and the fourth body portion 326 may be along the Z direction, but the present disclosure is not limited thereto.

In the above embodiment of the present disclosure, for example, an electronic element 37 and/or a cable module 36 may be disposed on the first body portion 323, the second body portion 324, the third body portion 325, and/or the fourth body portion 326. In addition, the light-emitting element 35 can be coupled to the flexible printed circuit board 32 and disposed on the light-emitting element carrier portion 322. Further, in the embodiment of the present disclosure, two light-emitting elements 35 may be provided, one of light-emitting element which may be disposed on the first carrier side portion 3221, and the other light-emitting element of which may be disposed on the second carrier side portion 3222. Additionally, one or more electronic elements 37 may be disposed on the first body portion 323 and/or the second body portion 324. The cables can mainly be arranged at the third body portion 325, which can assist the light-emitting element 35 in dissipating heat. The cable module 36 may be disposed on the fourth body portion 326 such that the fourth body portion 326 can act as a welding area for the connector 361 of the cable module 36. By using the fourth body portion 326 as the welding area of the connector 361 of the cable module 36, the convenience of soldering can thereby be improved. However, it should be noted that the present disclosure is not limited to the above examples.

As described above, referring to FIG. 1 to FIG. 3, the flexible printed circuit board 32 may further include a plurality of connecting portions 327. One of the connecting portions 327 may be connected between the sensing module carrier portion 321 and the first body portion 323, another one of the connecting portions 327 may be connected between the sensing module carrier portion 321 and the second body portion 324, yet another one of the connecting portions 327 may be connected between the first body portion 323 and the third body portion 325, still another one of the connecting portions 327 may be connected between the third body portion 325 and the light-emitting element carrier portion 322, and still another one of the connecting portions 327 may be connected between the second body portion 324 and the fourth body portion 326. Therefore, in the subsequent manufacturing process, the flexible printed circuit board 32 can be bent so as to bend the connecting portion 327, such that the flexible printed circuit board 32 can be windingly disposed around the carrier base 31.

As described above, referring to FIG. 1 to FIG. 3, the sensing module carrier portion 321 may include a first surface 3211 and a second surface 3212 corresponding to the first surface 3211 of the sensing module carrier portion 321. The light-emitting element carrier portion 322 may include a first surface 3223 and a second surface 3224 corresponding to the first surface 3223 of the light-emitting element carrier portion 322. The first body portion 323 may include a first surface 3231 and a second surface 3232 corresponding to the first surface 3231 of the first body portion 323. The second body portion 324 may include a first surface 3241 and a second surface 3242 corresponding to the first surface 3241 of the second body portion 324. The third body portion 325 may include a first surface 3251 and a second surface 3252 corresponding to the first surface 3251 of the third body portion 325. The fourth body portion 326 may include a first surface 3261 and a second surface 3262 corresponding to the first surface 3261 of the fourth body portion 326.

As described above, in the embodiment of the present disclosure, one surface (for example, the upper surface) of the flexible printed circuit board 32 may be formed jointly by the first surface 3211 of the sensing module carrier portion 321, the first surface 3223 of the light-emitting element carrier portion 322, the first surface 3231 of the first body portion 323, the first surface 3241 of the second body portion 324, the first surface 3251 of the third body portion 325, and the first surface 3261 of the fourth body portion 326. In addition, another surface (e.g., the lower surface) of the flexible printed circuit board 32 may be formed jointly by a second surface 3212 of the sensing module carrier portion 321, the second surface 3224 of the light-emitting element carrier portion 322, the second surface 3232 of the first body portion 323, the second surface 3242 of the second body portion 324, the second surface 3252 of the third body portion 325, and the second surface 3262 of the fourth body portion 326.

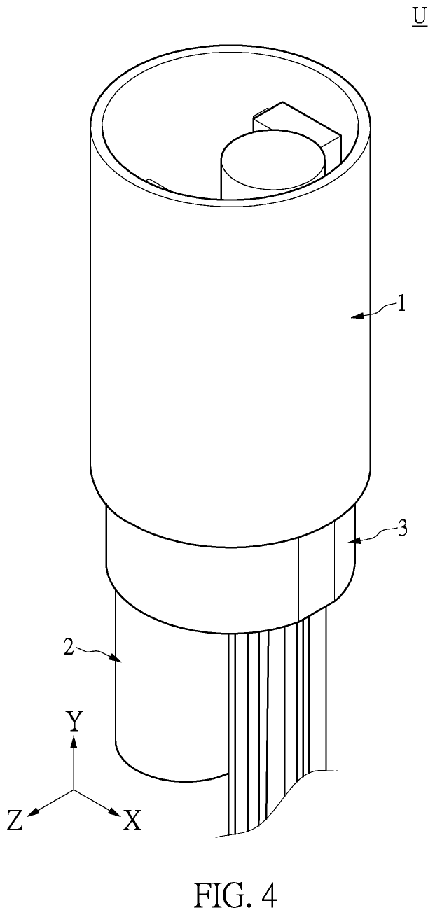

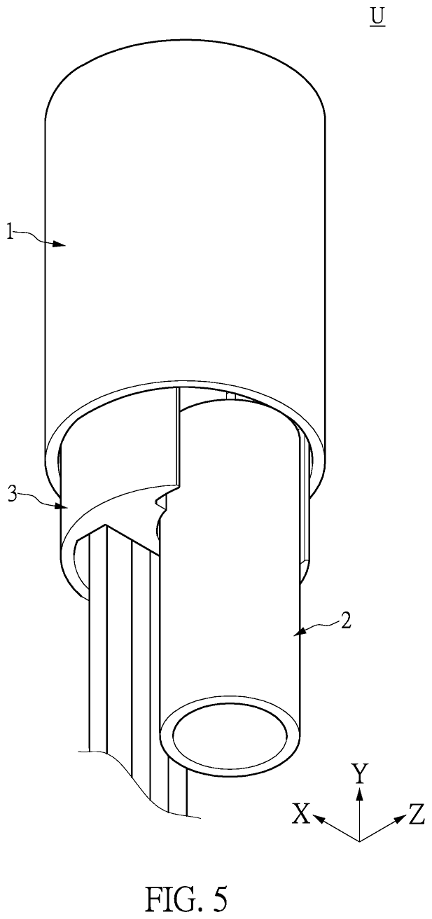

Next, referring to FIG. 4 to FIG. 7, FIG. 4 and FIG. 5 are perspective assembled views of an endoscope device according to the embodiment of the present disclosure. The components of the endoscope device U and the flexible printed circuit board assembly 3 provided by the embodiment of the present disclosure will be further described below. In detail, the endoscope device U may include a hollow tube 1 and a flexible printed circuit board assembly 3. In addition, in a preferred configuration of the present disclosure, the endoscope device U may further include a working channel 2. Further, the plurality of wires 362, the working channel 2 of the flexible printed circuit board assembly 3, and the cable module 36 may be disposed in the hollow tube 1, and the cable module 36 may be coupled to the flexible printed circuit board assembly 3. In addition, the working channel 2 can be used by medical instruments for access during operational procedures.

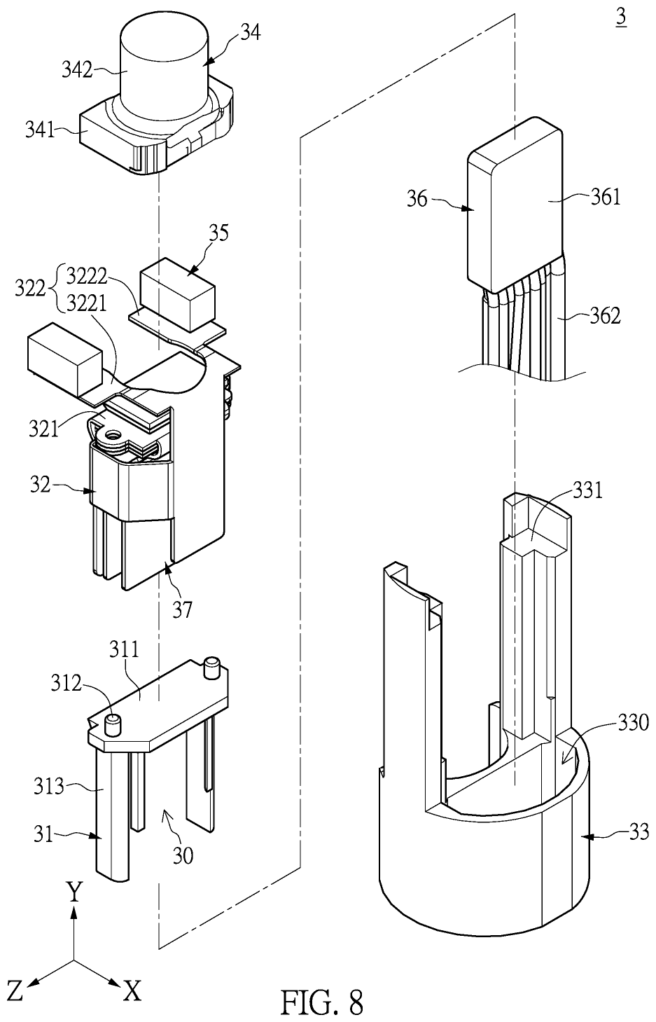

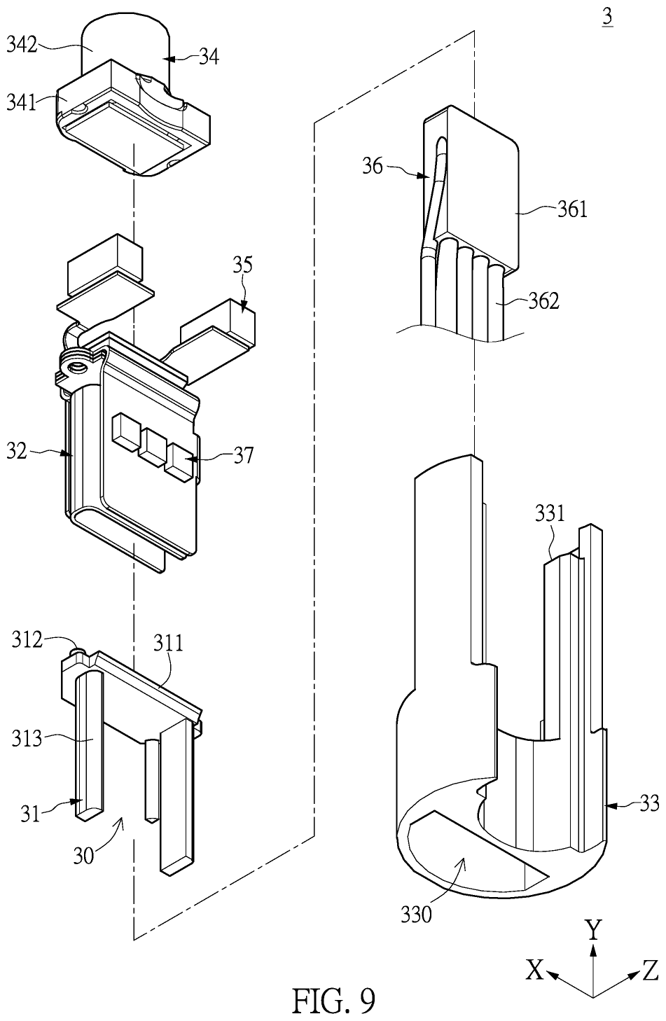

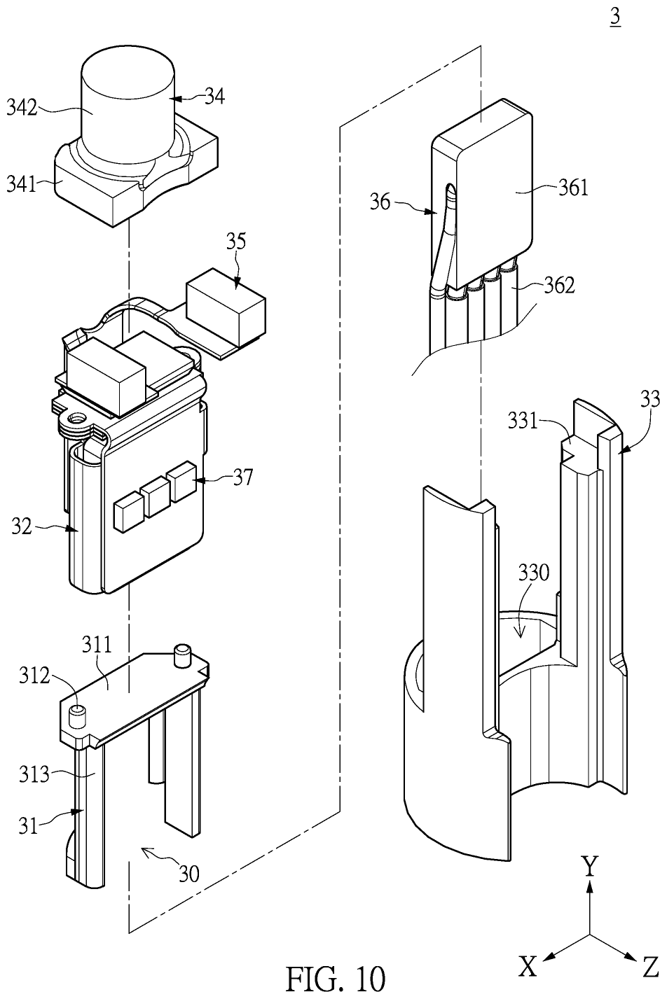

Next, referring to FIG. 8 to FIG. 10, FIG. 8 to FIG. 10 are perspective exploded views of a flexible printed circuit board assembly according to the embodiment of the present disclosure. The flexible printed circuit board assembly 3 may include a carrier base 31, a flexible printed circuit board 32, an image sensing module 34, a light-emitting element 35, and a cable module 36. The flexible printed circuit board 32 can be disposed on the carrier base 31. The image sensing module 34 can be coupled to the flexible printed circuit board 32 and disposed on a sensing module carrier portion 321 of the flexible printed circuit board 32. The light-emitting element 35 can be coupled to the flexible printed circuit board 32 and disposed on the flexible printed circuit board 32. The LED module 36 can be coupled to the flexible printed circuit board 32 and disposed on the flexible printed circuit board 32.

Referring to FIG. 8 to FIG. 10, and as shown in FIG. 6 and FIG. 7, the flexible printed circuit board assembly 3 may further include a supporting frame 33, and the carrier base 31 and the light-emitting element carrier portion 322 may be disposed on the supporting frame 33. The supporting frame 33 can be used to support the carrier base 31 and the light-emitting element carrier portion 322 such that the carrier base 31 and the light-emitting element carrier portion 322 are supported by the supporting frame 33. In addition, the supporting frame 33 may include a tank 330 and an abutting portion 331. The carrier base 31 may be disposed in the tank 330, and the light-emitting element carrier portion 322 may abut against the abutting portion 331. In addition, the flexible printed circuit board assembly 3 may further include a cable module 36. The cable module 36 may be coupled to the flexible printed circuit board 32. The cable module 36 may include a connector 361 coupled to the flexible printed circuit board 32 and a plurality of wires 362 disposed on the connector 361. The connector 361 can be coupled between the flexible printed circuit board 32 of the flexible printed circuit board assembly 3 and the plurality of wires 362. In addition, the flexible printed circuit board assembly 3 may further include at least one electronic element 37. The at least one electronic element 37 may be disposed on the flexible printed circuit board 32 and coupled to the flexible printed circuit board 32. In addition, the flexible printed circuit board assembly 3 may further include a holding element 328 (as shown in FIGS. 1 and 2). The holding element 328 may be disposed on the sensing module carrier portion 321 to serve as a supporting structure of the sensing module carrier portion 321. In addition, it should be noted that the coupling in the present disclosure may be a direct connection, an indirect connection, a direct electrical connection or an indirect electrical connection, and the present disclosure is not limited thereto.

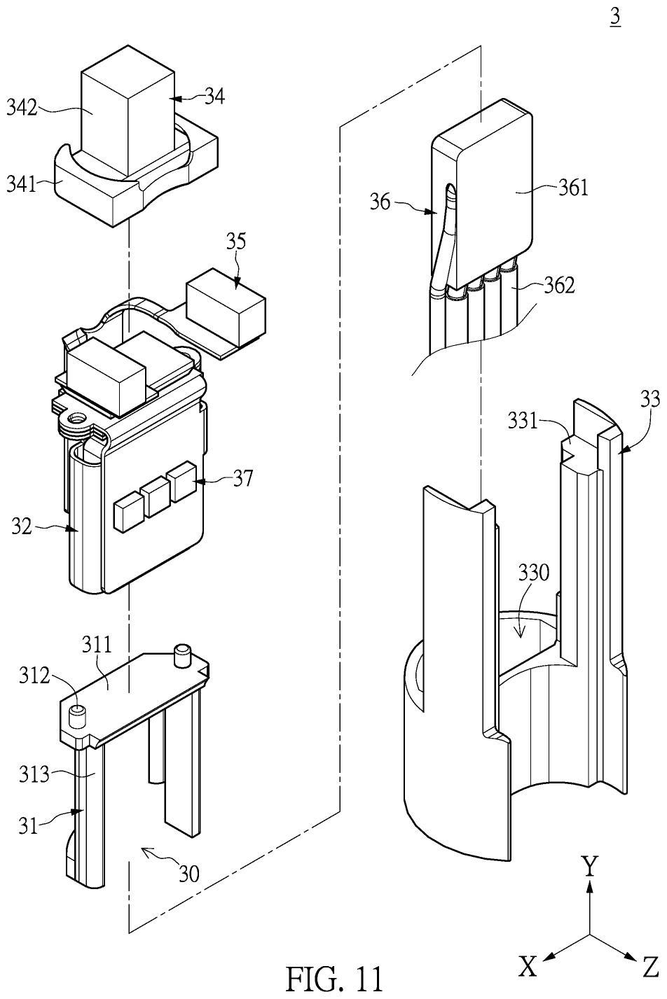

Referring to FIG. 8 to FIG. 10, and FIG. 11, FIG. 11 is a perspective exploded view of another aspect of a flexible printed circuit board assembly according to the embodiment of the present disclosure. For example, the image sensing module 34 can include a carrier 341 disposed on the sensing module carrier portion 321 and an image sensor 342 disposed on the carrier 341. In addition, as can be seen from the comparison of FIG. 8 to FIG. 10 and FIG. 11, the greatest difference between the configuration of FIG. 8 to FIG. 10 and that of FIG. 11 lies in the form of the image sensing module 34. The image sensor 342 of the image sensing module 34 as shown in FIG. 8 to FIG. 10 may include an optical sensor (not shown) and an optical element (e.g., a lens, not shown). Since the optical sensor and the optical element are two separate elements, the optical sensor and the optical element can be sequentially disposed on the carrier 341, and the optical sensor and the optical elements are aligned with each other by using the carrier 341 to achieve optical positioning. On the other hand, the image sensor 342 of the image sensing module 34 shown in FIG. 11 has an optical sensor (not shown) and an optical element (e.g., a lens, not shown) that are electronic elements integrated together; therefore, the carrier 341 can be optionally disposed. In other words, although the image sensing module 34 in FIG. 11 is disposed on the carrier 341, in other embodiments, the image sensing module 34 may be disposed directly on the flexible printed circuit board 32 without the carrier 341. In addition, the image sensor 342 can be, for example, but not limited to, a charge coupled device (CCD) or a complementary metal-oxide semiconductor (CMOS). In addition, it should be noted that the light-emitting element 35 can be, for example, a light-emitting diode (LED), and the number of the light-emitting elements 35 can also be one or more. However, the present disclosure is not limited thereto.

Next, referring to FIG. 1 to FIG. 3 and FIG. 8 to FIG. 10, the arrangement of the image sensing module 34, the light-emitting element 35, the cable module 36, the electronic element 37, and the holding element 328 will be further exemplified. In detail, the image sensing module 34 may be disposed on the first surface 3211 of the sensing module carrier portion 321. The light-emitting element 35 may be disposed on the first surface 3223 of the light-emitting element carrier portion 322. The holding element 328 may be disposed on the second surface 3212 of the sensing module carrier portion 321. The connector 361 of the cable module 36 may be disposed on the first surface 3261 of the fourth body portion 326. The electronic element 37 may be disposed on the first surface 3231 of the first body portion 323 and/or the first surface 3241 of the second body portion 324. The holding element 328 may be disposed on the second surface 3212 of the sensing module carrier portion 321. However, it should be noted that the present disclosure is not limited by the examples above.

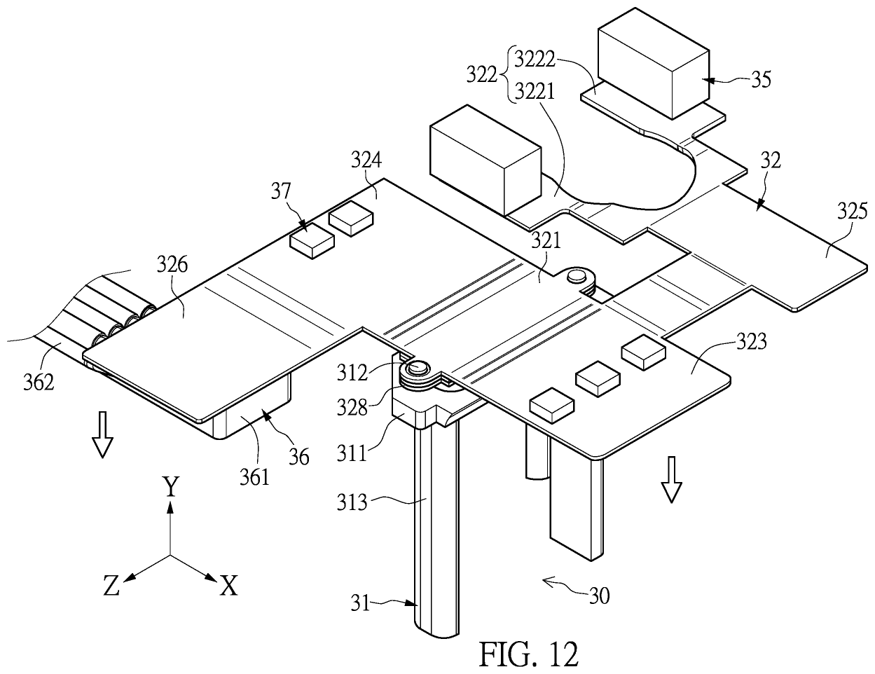

Then, referring to FIG. 1 to FIG. 3, FIG. 8 to FIG. 10 and FIG. 12 to FIG. 14, FIG. 12 to FIG. 14 are schematic diagrams of an assembly process of a flexible printed circuit board and a carrier base according to the embodiment of the present disclosure. In detail, in the embodiment of the present disclosure, the carrier base 31 may include a carrier board 311, one or more positioning posts 312 disposed on the carrier board 311, and a positioning frame 313 disposed on the carrier board 311. In the process of disposing the flexible printed circuit board 32 on the carrier base 31, the sensing module carrier portion 321 of the flexible printed circuit board 32 may first be abutted on the carrier board 311. In the embodiment of the present disclosure, the sensing module carrier portion 321 of the flexible printed circuit board 32 may further include one or more positioning holes 3210, and the positioning post 312 of the carrier base 31 may be disposed through the positioning hole 3210 of the flexible printed circuit board 32. Thereby, the positional relationship between the flexible printed circuit board 32 and the carrier base 31 is fixed. Further, the holding element 328 disposed on the second surface 3212 of the sensing module carrier portion 321 may also include one or more positioning holes 3280, and the positioning hole 3280 of the holding element 328 may correspond to the positioning hole 3210 of the flexible printed circuit board 32 so that the positioning post 312 of the carrier base 31 can be disposed through the positioning hole 3210 of the flexible printed circuit board 32 and the positioning hole 3280 of the holding element 328.

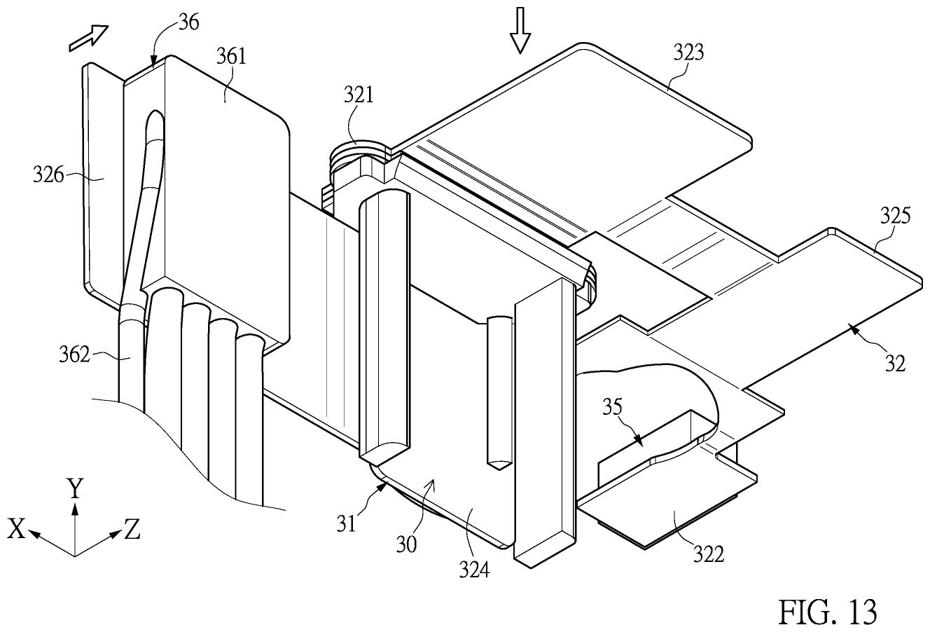

As shown in FIG. 12 and FIG. 13, after the sensing module carrier portion 321 of the flexible printed circuit board 32 is abutted against the carrier board 311, the second body portion 324 connected to the side of the sensing module carrier portion 321 can be bent downward so that the connecting portion 327 connected between the sensing module carrier portion 321 and the second body portion 324 is bent. After the second body portion 324 of the flexible printed circuit board 32 is bent downward, the fourth body portion 326 connected to the second body portion 324 may further be bent forward so that the connecting portion 327 connected between the second body portion 324 and the fourth body portion 326 is bent and abuts against the other side of the positioning frame 313. Thereby, the connector 361 of the cable module 36 disposed on the fourth body portion 326 can be located in an accommodating space 30.

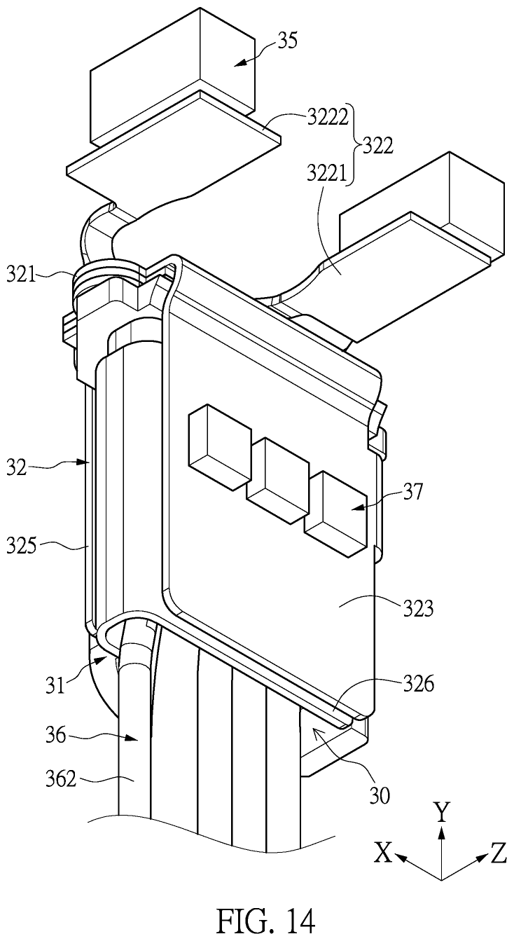

After that, as shown in FIG. 13 and FIG. 14, after the connector 361 of the cable module 36 disposed on the fourth body portion 326 is located in the accommodating space 30, the first body portion 323 connected to the side of the sensing module carrier portion 321 can be bent downward so that the connecting portion 327 connected between the sensing module carrier portion 321 and the first body portion 323 is also bent. Thereby, the first surface 3231 of the first body portion 323 faces the first surface 3241 of the second body portion 324. Next, the third body portion 325 connected to the first body portion 323 may be bent backward so that the connecting portion 327 connected between the first body portion 323 and the third body portion 325 is bent and abuts against one side of the positioning frame 313. Thereby, the second body portion 324 is located between the first body portion 323 and the third body portion 325, and the second surface 3252 of the third body portion 325 faces the first surface 3241 of the second body portion 324. Further, the fourth body portion 326 may be located between the first body portion 323 and the second body portion 324 so that the first surface 3261 of the fourth body portion 326 faces the second surface 3232 of the first body portion 323. Further, when the fourth body portion 326 is bent forward so that the fourth body portion 326 is located between the first body portion 323 and the second body portion 324, and when the third body portion 325 is bent backward so that the second body portion 324 is located at the first body portion 323 and the third body portion 325, the first carrier side portion 3221 and the second carrier side portion 3222 connected to the third body portion 325 may be bent forward. Thereby, the flexible printed circuit board 32 can be disposed in a curved shape on the carrier base 31, the accommodating space 30 can be defined between the curved flexible printed circuit board 32 and the carrier base 31, and the connector 361 of the cable module 36 can be disposed in the accommodating space 30. In addition, the image sensing module 34 can be located between one of the light-emitting elements 35 and the other one of the light-emitting elements 35, and the light projection direction (Y direction) of the light-emitting element 35 is the same as the image capturing direction of the image sensing module 34 (Y direction).

In conclusion, one of the beneficial effects of the present disclosure is that, the endoscope device U, the flexible printed circuit board assembly 3 and the flexible printed circuit board 32 thereof provided by the present disclosure has the technical feature of “the flexible printed circuit board 32 being disposed in a curved shape on the carrier base 31, an accommodating space 30 being defined between the curved flexible printed circuit board 32 and the carrier base 31, and the connector 361 of the cable module 36 being coupled to the flexible printed circuit board 32 and disposed in the accommodating space 30,” so as to improve the heat dissipation efficiency and enable electronic elements to be configured at appropriate positions.

Further, by using the fourth body portion 326 as the welding area of the connector 361 of the cable module 36, the convenience of soldering can be improved.

Further, the positioning post 312 of the carrier base 31 may be disposed through the positioning hole 3210 of the flexible printed circuit board 32. Thereby, the flexible printed circuit board 32 and the carrier base 31 can have a more stable connection therebetween.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An endoscope device, comprising:
a hollow tube; and
a flexible printed circuit board assembly disposed in the hollow tube, including a carrier base, a flexible printed circuit board, an image sensing module, an light-emitting element, a cable module, and a supporting frame;
wherein the flexible printed circuit board is disposed on the carrier base, the image sensing module is coupled to the flexible printed circuit board and disposed on a sensing module carrier portion of the flexible printed circuit board, the light-emitting element is coupled to the flexible printed circuit board and disposed on an light-emitting element carrier portion of the flexible printed circuit board, the cable module is coupled to the flexible printed circuit board and disposed on the flexible printed circuit board, and the supporting frame is configured to support the carrier base and the light-emitting element carrier portion;
wherein the flexible printed circuit board is disposed in a curved shape on the carrier base, an accommodating space is defined between the flexible printed circuit board and the carrier base, and a connector of the cable module is coupled to the flexible printed circuit board and disposed in the accommodating space;
wherein, the flexible printed circuit board includes a sensing module carrier portion, a first body portion, a second body portion, a third body portion, a fourth body portion and a light-emitting element carrier portion, the first body portion is connected to one side of the sensing module carrier portion, the second body portion is connected to the other side of the sensing module carrier portion and corresponds to the first body portion, the third body portion is connected to the first body portion, the fourth body portion is connected to the second body portion and the light-emitting element carrier portion is connected to the third body portion;
wherein, the accommodating space is defined between the fourth body portion and the carrier base, and the connector of the cable module is disposed on the fourth body portion and located in the accommodating space.

2. The endoscope device according to claim 1, further comprising: a working channel disposed in the hollow tube.

3. The endoscope device according to claim 1, wherein the cable module further includes a plurality of wires disposed on the connector and coupled to the connector.

4. A flexible printed circuit board assembly, comprising:
a carrier base;
a flexible printed circuit board disposed on the carrier base, including a sensing module carrier portion, a first body portion, a second body portion, a third body portion, and an light-emitting element carrier portion, wherein the first body portion is connected to one side of the sensing module carrier portion, the second body portion is connected to the other side of the sensing module carrier portion and corresponds to the first body portion, the third body a portion is connected to the first body portion, and the light-emitting element carrier portion is connected to the third body portion;
an image sensing module coupled to the flexible printed circuit board and disposed on the sensing module carrier portion;
an light-emitting element coupled to the flexible printed circuit board and disposed on the light-emitting element carrier portion; and
a cable module coupled to the flexible printed circuit board, including a connector coupled to the flexible printed circuit board and a plurality of wires disposed on the connector;
wherein the flexible printed circuit board is disposed in a curved shape on the carrier base, an accommodating space is defined between the flexible printed circuit board and the carrier base, and the connector of the cable module is disposed in the accommodating space;
wherein the flexible printed circuit board further comprises a fourth body portion coupled to the second body portion, wherein the accommodating space is defined between the fourth body portion and the carrier base, and the connector of the cable module is disposed on the fourth body portion and located in the accommodating space.

5. The flexible printed circuit board assembly according to claim 4, further comprising: a supporting frame, the carrier base and the light-emitting element carrier portion being disposed on the supporting frame and supported by the supporting frame.

6. The flexible printed circuit board assembly according to claim 4, further comprising: at least one electronic element disposed on the first body portion, the second body portion, or the third body portion.

7. The flexible printed circuit board assembly according to claim 4, further comprising: a holding element disposed on the flexible printed circuit board.

8. The flexible printed circuit board assembly according to claim 4, wherein the light-emitting element carrier portion includes a first carrier side portion connected to the third body portion and a second carrier side portion connected to the third body portion, and the first carrier side portion and the second carrier side portion are separated from each other.

9. The flexible printed circuit board assembly according to claim 9, further comprising: another light-emitting element, wherein one of the light-emitting elements is disposed on the first carrier side portion, the other one of the light-emitting elements is disposed on the second carrier, and the image sensing module is located between the two light-emitting elements.

10. The flexible printed circuit board assembly according to claim 4, wherein the image sensing module includes a carrier disposed on the sensing module carrier portion and an image sensor disposed on the carrier.

11. The flexible printed circuit board assembly according to claim 4, wherein the sensing module carrier portion includes a first surface and a second surface corresponding to the first surface of the sensing module carrier portion, and the image sensing module is disposed on the first surface of the sensing module carrier portion; wherein the light-emitting element carrier portion includes a first surface and a second surface corresponding to the first surface of the light-emitting element carrier portion, and the light-emitting element is disposed on the first surface of the light-emitting element carrier portion.

12. The flexible printed circuit board assembly according to claim 11, further comprising: a holding element disposed on the second surface of the sensing module carrier portion.

13. The flexible printed circuit board assembly according to claim 11, wherein the second body portion is located between the first body portion and the third body portion, the first body portion includes a first surface and a second surface corresponding to the first surface of the first body portion, the second body portion includes a first surface and a second surface corresponding to the first surface of the second body portion, and the third body portion includes a first surface and a second surface corresponding to the first surface of the third body portion; and wherein the first surface of the first body portion faces the first surface of the second body portion, and the first surface of the third body portion faces the second surface of the second body portion.

14. The flexible printed circuit board assembly according to claim 4, wherein the fourth body portion includes a first surface and a second surface corresponding to the first surface of the fourth body portion, the connector of the cable assembly being disposed on the first surface of the fourth body portion.

15. The flexible printed circuit board assembly according to claim 4, wherein the carrier base includes a carrier board, a positioning post disposed on the carrier board, and a positioning frame disposed on the carrier board; wherein the sensing module carrier portion of the flexible printed circuit board is abutted on the carrier board; wherein the sensing module carrier portion of the flexible printed circuit board further includes a positioning hole, and the positioning post of the carrier base is disposed through in the positioning hole of the flexible printed circuit board; wherein the flexible printed circuit board is windingly disposed around the carrier board and the positioning frame of the carrier base.

16. A flexible printed circuit board, comprising:
a sensing module carrier portion;
a first body portion connected to one side of the sensing module carrier portion;
a second body portion connected to the other side of the sensing module carrier portion and corresponding to the first body portion, wherein the first body portion and the second body portion are respectively located on opposite sides of the sensing module carrying portion;
a third body portion coupled to the first body portion;
a fourth body portion coupled to the second body portion, and the third body portion and the fourth body portion are respectively located on opposite sides of the sensing module carrying portion; and
an light-emitting element carrier portion, the light-emitting element carrier portion being coupled to the third body portion;
wherein the light-emitting element carrier portion includes a first carrier side portion connected to the third body portion and a second carrier side portion connected to the third body portion, and the first carrier side portion and the second carrier side portion are separated from each other;
wherein the extending directions of the first body portion and the second body portion may be substantially perpendicular to the extending directions of the third body portion and the fourth body portion.
